(19) United States
(12) Patent Application Publication
(10) Pub. No.: US 2009/0287089 A1
(43) Pub. Date: Nov. 19, 2009

(54) Methods, devices and apparatus for imaging for reconstructing a 3-D image of an area of interest

(75) Inventor: Peter Spector, Burlington, VT (US)

Correspondence Address: EDWARDS ANGELL PALMER & DODGE LLP, P.O. BOX 55874, BOSTON, MA, 02205, US

(73) Assignee: The University of Vermont and State Agriculture College, Burlington, VT (US)

(21) Appl. No.: 12/322,383

(22) Filed: Feb. 2, 2009

Related U.S. Application Data
(60) Provisional application No. 61/025,097, filed on Jan. 31, 2008.

(51) Int. Cl. A61B 8/00

(52) U.S. Cl. 600/466

(57) ABSTRACT

Featured are methods, devices and apparatuses for imaging tissue, such as cardiac tissue, using any of a number of imaging techniques (e.g., ultrasonic imaging techniques) and a 3-D tracking system. In embodiments, such methods, devices and apparatuses are configured so that a 3-D image can be reconstructed from image data acquired at different locations from the image data and three dimensional coordinates that are determined for each location whereat the imaging device was located.

This application claims the benefit of U.S. Provisional Application Ser. No. 61/025,097 filed Jan. 31, 2008, the teachings of which are incorporated herein by reference.

### FIELD OF INVENTION

The present invention generally relates to methods and systems for imaging using ultrasonic imaging and tracking techniques and more particularly for such imaging and tracking systems so as to allow registration of multiple echo views with each other to reconstruct a 3-D image.

### BACKGROUND OF THE INVENTION

Cardiac arrhythmias, and atrial fibrillation in particular, persist as common and dangerous medical aliments associated with abnormal cardiac chamber wall tissue, and are often observed in elderly patients. In patients with cardiac arrhythmia, abnormal regions of cardiac tissue do not follow the synchronous beating cycle associated with normally conductive tissue in patients with sinus rhythm. Instead, the abnormal regions of cardiac tissue aberrantly conduct to adjacent tissue, thereby disrupting the cardiac cycle into an asynchronous cardiac rhythm. Such abnormal conduction is known to occur at various regions of the heart, such as, for example, in the region of the sino-atrial (SA) node, along the conduction pathways of the atrioventricular (AV) node and the Bundle of His, or in the cardiac muscle tissue forming the walls of the ventricular and atrial cardiac chambers.

Typically to treat such cardiac arrhythmias, including atrial arrhythmia, one images the area of interest using any of a number of techniques known to those skilled in the art. Such imaging usually involves navigating a probe (e.g., a catheter, endoscope) from a point of entry in the body to a location where the heart can be imaged. Also, in various techniques for treating cardiac arrhythmias a catheter is used, which is navigated from a point of entry in the body's circulatory system to generally the location of the heart. It should be recognized that there are a number of medical procedures known to those skilled in the art, in which the area of interest is imaged using any of a number of imaging techniques known to those skilled in the art for purposes of diagnosis and/or treatment.

Generally described in U.S. Pat. No. 5,797,849, U.S. Pat. No. 6,246,898 and WO 99/58055 (PCT/US99/10125) are methods for carrying out a medical procedure using a 3-D ultrasonic tracking and imaging system. As described, a surgical instrument, such as a catheter, probe, sensor, needle or the like is inserted into a living being, and the position of the surgical instrument is tracked as it moves through a medium in a bodily structure. The location of the surgical instrument relative to its immediate surroundings is displayed to improve a physician's ability to precisely position the surgical instrument. The medical procedures include: removal of an obstruction from the circulatory system, a biopsy, amniocentesis, brain surgery, measurement of cervical dilation, evaluation of knee stability, assessment of myocardial contractibility, eye surgery, prostate surgery and transmyocardial myocardial revascularization (TMR). In addition, the described method also finds use in connection with the generation of 2-D echo planes.

In connection with cardiac related medical procedures, such methods have been embodied in an intracardiac ultrasound catheter, which allows non-contact chamber mapping without requiring the placement of a catheter in that chamber. This allows for the rapid accumulation of data for creation of anatomical shells, while at the same time there is no deformation of the chamber being studied and there is improved safety as the catheter is not placed in the Aorta. Such a system, however, is less sensitive to structures that are vertical (e.g., the ridge between the LAA and the LSPV).

Such a 3-D tracking and imaging system can work with many individual transducers that can be energized sequentially at very high repetition rates, thereby giving the impression that several distances are being measured instantaneously. In reality, the distances are measured in sequence, but since the delay time between successive measurements is on the order of 100 microseconds, the measurements occur virtually simultaneously for most biological applications.

It thus would be desirable to provide new devices and methods that would allow a clinician to perform a 3D reconstruction of any structure, more particularly to allow for the enhancement of diagnostic ultrasound independent of electro-anatomic mapping. Preferably such devices and methods would allow a clinician to obtain views of the structure or at least parts of the structure from a plurality of different anatomical locations that can be referenced to each other using the same 3-D tracking system.

### SUMMARY OF THE INVENTION

In an aspect of the present invention there is featured a method, devices and apparatus for imaging tissue, such as cardiac tissue, using ultrasonic imaging techniques and a 3-D ultrasonic tracking system. Such a method includes mounting at least one transducer (at least one mobile transducer) to a probe (e.g., an ultrasonic probe) and mounting one or more reference ultrasonic transducers at locations on/in and about the body to be imaged, where the one or more reference transducers each have a position fixed relative to the body during/throughout the imaging process. In more particular embodiments, such a method further includes mounting a plurality of reference ultrasonic transducers at various body locations, where the plurality of reference transducers each have a position fixed relative to the body during/throughout the imaging process. In more specific embodiments, three or more or four or more reference ultrasonic transducers are mounted at various body locations.

Such a method further includes generating three dimensional coordinates of the at least one mobile ultrasonic transducer on the probe relative to a reference frame established by the reference ultrasonic transducers, generating image data using the probe or ultrasonic probe and registering the three-dimensional coordinates with the image data from the probe to form a 3-D image scene.

In further embodiments, such a method includes performing said steps of generating three dimensional coordinates, generating image date for a given body part and registering when the probe is located at a first location, and performing said steps of generating three dimensional coordinates, generating image date for the given part and registering when another probe is located at a second body location, the generating of three dimensional coordinates at the first and second locations includes using the same one or more reference transducers and the second location being remote from the first location. In yet further embodiments, said steps of generating three dimensional coordinates, generating image date for given body part and registering are performed N times, at N different locations of the ultrasonic probe.

It should be recognized that in further embodiments, the different locations are selected so that a given body part is imaged from different vantage points. In exemplary embodiments, the body part is the heart and the heart is imaged from one or more locations using a catheter ultrasonic probe that is located in the circulatory system and is imaged from yet another second location or vantage point that is remote from the one or more locations in the circulatory system. In further illustrative embodiments, a trans-esophageal ultrasonic probe on which is mounted the at least one transducer is located within the esophagus for imaging of the heart. In a trans-esophageal echo cardiography (TEE) procedure, a ultrasonic transducer is attached to an endoscope (e.g., a fiber optic endoscope) that is introduced into the esophagus and the ultrasonic waves are employed to obtain image data of the heart chambers, valves and surrounding structures.

As an intracardiac ultrasound catheter is not as easily rotated in the horizontal plane as the vertical due to secondary physical restrictions, it is less sensitive to structures that are vertical. In the present invention, the trans-esophageal ultrasonic probe used in a TEE procedure has a greater ability in comparison to be rotated in the horizontal plane. As the three-dimensional location of the trans-esophageal ultrasonic probe also is referenced to the same external reference transducers, the methodology of the present invention provides a more robust application as it enhances image mapping because the image data acquired at the second location can be combined with other acquired image data and used to perform a 3D reconstruction on any structure being imaged as all acquired image data have a common frame of reference.

Other aspects and embodiments of the invention are discussed below.

### BRIEF DESCRIPTION OF THE DRAWING

For a fuller understanding of the nature and desired objects of the present invention, reference is made to the following detailed description taken in conjunction with the accompanying drawing figures wherein like reference character denote corresponding parts throughout the several views and wherein:

FIG. 1 is a high level flow diagram of the methodology of the present invention.

FIG. 2 is an illustrative view showing mounting of the reference ultrasonic transducers at different body locations;

FIG. 3 is a schematic representation of four transducers in three dimensional space, for tracking an triangulating the three dimensional positions of each transducer; and

FIG. 4 is an illustrative block diagram of an illustrative 3-D tracking and imaging system.

### DESCRIPTION OF THE PREFERRED EMBODIMENT

The present invention features methods, devices and apparatuses for imaging tissue, such as cardiac tissue, using ultrasonic imaging techniques and a 3-D ultrasonic tracking system. It also is within the scope of the present invention, to use image data acquired using other imaging modalities and reference such other image data to the reference frame of the external transducers so that such other data is used to reconstruct the three-dimensional image of the area of interest (e.g., heart).

Such systems and methods allows for non-contacting imaging or mapping of a body part such as for example the heart. More particularly, such systems and methods allow a user a to acquire image data at two or more anatomical locations and using the three-dimensional location of where the image data was acquired to reconstruct a three dimensional image of the area of interest using the image date that was acquired at the two or more locations.

For example, in exemplary embodiments, the body part is the heart and the heart is imaged from one or more locations using a catheter ultrasonic probe (e.g., an intracardiac ultrasound catheter) that is located in the circulatory system and is imaged from yet another second location or vantage point that is remote from the one or more locations in the circulatory system. In further illustrative embodiments, a trans-esophageal ultrasonic probe is located within the esophagus for imaging of the heart. In a trans-esophageal echo cardiography (TEE) procedure, a ultrasonic transducer is attached to an endoscope (e.g., a fiber optic endoscope) that is introduced into the esophagus and the ultrasonic waves are employed to obtain image data of the heart chambers, valves and surrounding structures.

An intracardiac ultrasound catheter is not as easily rotated in the horizontal plane as the vertical due to secondary physical restrictions, and thus is less sensitive to structures that are vertical. The trans-esophageal ultrasonic probe used in a TEE procedure on the other hand has a greater ability in comparison to be rotated in the horizontal plane. As the three-dimensional location of the trans-esophageal ultrasonic probe and the location of the intracardiac ultrasound catheter are both referenced to the same external frame of reference, the methodology of the present invention provides a more robust application. It enhances image mapping because the image data acquired at all different image acquisition locations is combined and used to perform a 3D reconstruction on any structure being imaged.

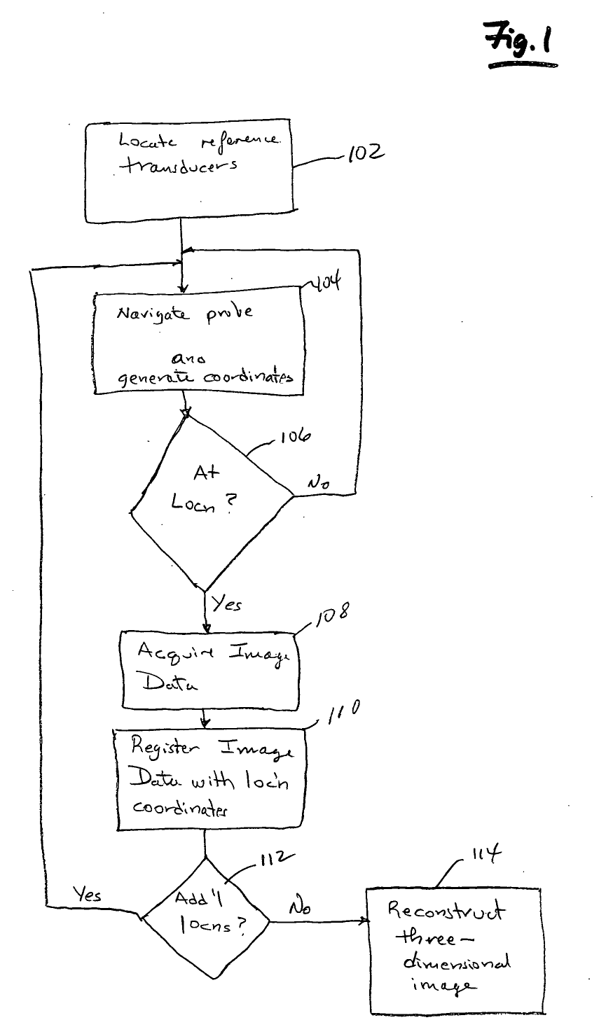

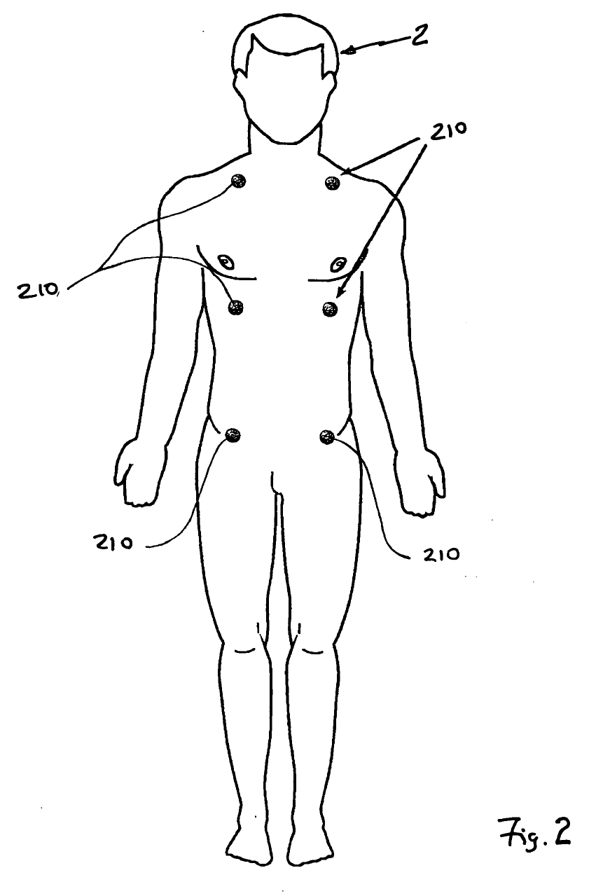

Referring now to FIG. 1 there is shown a high level flow diagram that illustrates an exemplary methodology of the present invention. Reference also should be made to FIGS. 2-4, for other details of the present invention such as placement of the reference transducers 210 and the three-dimensional (3-D) 3-D tracking and imaging system 200 of the present invention. Reference also should be made to the teachings in U.S. Pat. No. 5,797,849, U.S. Pat. No. 6,246,898 and WO 99/58055 (PCT/US99/10125), for example (the teachings of which are incorporated herein by reference), to the 3-D (three dimensional) ultrasonic tracking system described therein that uses one or more reference ultrasonic transducers at locations on the body to be imaged, where the one or more reference transducers each have a position fixed relative to the body during the imaging process.

In accordance with the methods of the present invention, the individual, body 2 or object to be imaged is initialed prepared for the particular imaging modality or modalities being used to image the area of interest. Such actions also include locating the individual, body 2 or object (hereinafter “body” 2) on a table, platform or other surface used in connection with the imaging process.

After preparing the body 2 for the imaging process, the clinician, technician or medical personnel, locate and mount one or more ultrasonic transducers, one or more reference transducers 210 on and/or in and about the body, Step 102. In more particular embodiments, the clinician, technician or medical personnel, locate and mount a plurality of reference transducers 210 at various body locations, where the plurality of reference transducers each have a position fixed relative to the body during/throughout the imaging process. In more specific embodiments, the clinician, technician or medical personnel, locate and mount X reference transducers are at various body locations, where X is an integer that satisfies one of the following relationships: X≧3 and X≧4. In yet more specific embodiments, the clinician, technician or medical personnel, locate and mount a multiplicity of reference, transducers are at various body locations.

Each of the reference transducers are located on the body 2, such as illustratively shown in FIG. 2, so that the 3-D tracking and imaging system 200 can determine the three-dimensional location of the probe or instrument 202 being used in connection with the particular medical procedure being performed, in particular. In particular, each of the different locations planned for use in the medical procedure. Also, the reference transducers 210 are mounted to or in the body 2 using any of a number of techniques known to those skilled in the art or hereinafter developed (e.g., adhesives) by which the transducer is maintained in fixed relation to the body 2 during relevant portions of the medical procedure, also as shown in FIG. 2. In addition to the reference transducers 210, the probe or instrument 202 is configured so as to include one or more ultrasonic transducers, one or more mobile transducers 212.

In an illustrative exemplary embodiment, a reference transducer 210 is a cylindrical piezoelectric ceramic crystal, encapsulated with an electrically insulating sealant. In further illustrative embodiments, each reference transducer is a part of an individual receiver circuit that includes a step-up isolation transformer, a two stage amplifier collectively providing a gain, a linear operational amplifier, a half-wave rectifier and a TTL level inverter. The digital waveform output from the TTL inverter is further isolated using an RF choke before it is transmitted back through a shielded cable.

In illustrative exemplary embodiments, the mobile transducer 212 can take many forms. According to an exemplary embodiment, a cylindrical or ring shaped ultrasonic transducer is provided for attachment to an instrument (e.g., a catheter or other probe), for the purpose of tracking its position in three dimensions inside the body or organ. The transducers can be either rigid or flexible. If they are rigid, they are typically constructed from PZT material, and cast or milled into an appropriate shape. If the transducers are made flexible, they are typically constructed from PVDF material that is laminated onto the surface of an instrument. This material is flexible and can be applied to rounded surfaces. As a result of the relatively low transmit efficiency of PVDF material, it is likely to be used for a transducer used in receive mode only. Appropriate surfaces of the transducers are coated with a conductive material to facilitate connection to leads. If the material is poled through the wall thickness, then the inner and outer surfaces of the cylinder are plated and conductors are affixed thereto.

While the size of the transducer will depend on the application of the tracking technology, the inner diameter of the cylinder is typically 5 millimeters or less for catheters and 5 millimeters or more for larger endoscopic probes. It should also be appreciated that several sections of a cylinder may be placed around the instrument of interest, thus making each individual transducer small for ease of manufacture, mounting or to control resonant frequency.

After locating and mounting the transducers (Step 102), the clinician or medical personnel, navigates the probe from the point of insertion and generates three-dimensional coordinates of the probe using the array of reference transducers 210 and the one or more mobile transducers 212, Step 104. Such a step includes the clinician or medical personnel taking the appropriate steps for inserting the probe into or on the body 2. For example, inserting a catheter including the probe into a vein or artery which is navigated through the circulatory system as is know to those skilled in the art. or inserting an endoscope or other device or apparatus through a natural (e.g., esophagus) or artificial body opening.

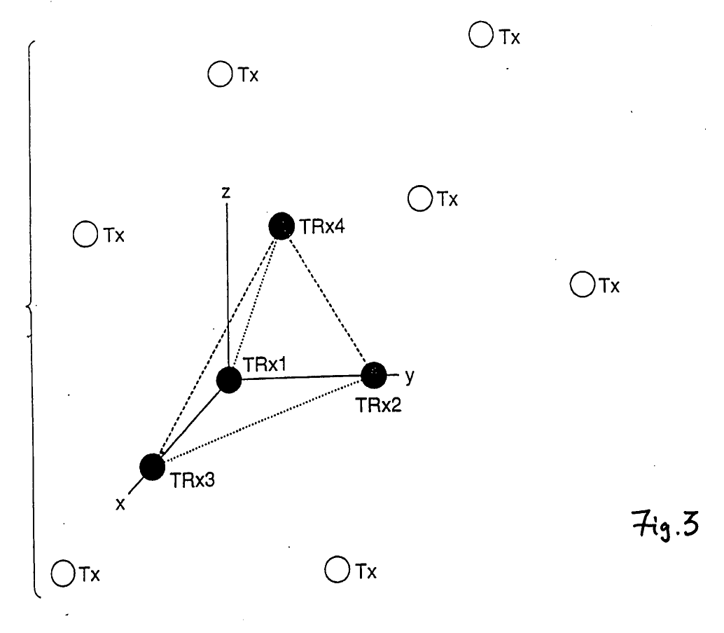

As discussed herein, the 3-D tracking and imaging system 200 of the present invention utilizes a plurality of transceivers, each of which can be programmed to operate as a transmitter or a receiver. By utilizing four or more transceivers, full three-dimensional measurement capability is provided, as shown in FIG. 3. Any three transceivers (TRx1, TRx2 and TRx3) lay in a plane (i.e., the x,y plane). The fourth transceiver (TRx4) can then be used to determine the z coordinates of the surrounding transducers (i.e., multiple crystals Tx) by determining if an active one of the transmitter transducers lies above or below the reference plane established by transceivers TRx1, TRx2 and TRx3. Each of the many transmitters (Tx) attached to the specimens are sequentially fired, while all reference transceivers record the receiver signals. Because the distance from each transmitter to the reference plane created by the transceivers is known, the relative x, y, z, coordinates of the transmitters can be determined. This is done in real time on a personal computer (PC) with the use of triangulation. This method of networking the transducers is unique to the 3-D tracking portion of the 3-D tracking and imaging system 200, and permits the user to trace the three-dimensional motion of the probe or instrument 202.

In a particular exemplary embodiment, except for the reference and mobile transducers 210,212, a portion of the 3-D tracking and imaging system 200 is embodied as an applications program that is executed on a personal computer as is known to those skilled in the art or as extension cards embodied in such a computer that are operably coupled to the operating system of such a computer. Due to the speed of the controlling computer, such a tracking system is capable of detecting small distance increments, for example, as small as 19 μm. The acquired data can be displayed on the display or computer screen as it is being obtained, and thus also can be saved to the computer's storage media automatically or by a simple key stroke. Obviously, the greater the number of transmitters, the better is the reconstruction.

As such a 3-D tracking and imaging system 200 can work with many individual transducers that can be energized sequentially at very high repetition rates, this gives the impression that several distances are being measured instantaneously. While the distances are measured in sequence; as the delay time between successive measurements is on the order of 100 microseconds, the measurements occur virtually simultaneously for most biological applications.

Such tracking and position determination are continued until the probe or instrument 202 reaches the desired location for imaging the area of interest, Step 106, No). When it is determined that the probe or instrument 202 has reached the desired location for imaging (Step 106, Yes), the clinician, technician or medical personnel starts the process involved with acquiring imaging data using the desired imaging modality, and continues the process until all of the desired image data is acquired, Step 108.

For example, if the instrument 202 or probe is an ultrasonic probe or instrument, by mounting the at least one mobile transducer 212 to the tip of the ultrasonic probe, and externally mounting the references transducers 210 to the patient/body, a 3-D tracking and imaging system 200 such as that described herein, can track the 3-D position of the imaging tip of the ultrasonic probe or instrument.

Typically, such a 3-D tracking and imaging system 200 records the 3-D position of the tip of the ultrasonic probe as well as any of its other positional information (e.g., angular orientation) along with the acquired ultrasonic image data being saved and/or acquired. This information is later used later when reformatting and spatially locating the acquired images to reconstruct the 3-D image of the area of interest.

After acquiring the image data, the image data is registered with respect to the 3-D coordinates at which the probe or instrument was located, Step 110. As indicated herein, such information is later used to reconstruct the three-dimensional image of the area of interested being imaged using the imaging modality. Thereafter, a determination is made as to whether or not additional imaging is to be performed at a different location (e.g., a different anatomical location) that is remote from the prior imaging location(s), Step 112.

If it is determined that additional imaging is to be performed at a different location or second location (Step 112, Yes) then the methodology further includes performing said step of navigating another probe and generating three dimensional coordinates (Step 104), generating image date for a given body part or a portion when it is determined that the probe or instrument is at the second or another location (Steps 106, 108), and registering the image date for the given body part with the three dimensional coordinates of the second or another body location (Step 110). It should be recognized that the probe or instrument 202 at the first imaging location also is withdrawn from the body 202 as part of the navigating step.

As described herein, the three-dimensional coordinates at each of the first location and the second/another location are determined using the same one or more reference transducers 210. In other words, the reference transducers remain in fixed relation to each other and the body 202, during each of the imaging processes. Each new location used for imaging, is remote from each of the prior locations used for imaging. In yet further embodiments, said steps being repeated are performed n times, at n different locations of the ultrasonic probe, where n is greater than or equal to two (n≧2). It should be recognized that in yet further embodiments, the different locations are selected so that the given body part or portion thereof is imaged from different vantage points.

In exemplary embodiments, the body part is the heart and the heart image is imaged from one or more locations using a catheter ultrasonic probe that is located in the circulatory system and is imaged from yet another second location or vantage point that is remote from the one or more locations in the circulatory system.

In further illustrative embodiments, a trans-esophageal ultrasonic probe on which is mounted at least one transducer is located within the esophagus for imaging of the heart from the esophagus. In a trans-esophageal echo cardiography (TEE) procedure, a ultrasonic transducer is typically attached to an endoscope (e.g., a fiber optic endoscope) which is introduced into the esophagus. The ultrasonic waves from the transducer are employed to obtain image data of the heart chambers, valves and surrounding structures.

As an intracardiac ultrasound catheter is not as easily rotated in the horizontal plane as the vertical due to secondary physical restrictions, it is less sensitive to structures that are vertical. In the present invention, the trans-esophageal ultrasonic probe used in a TEE procedure has a greater ability, in comparison, to be rotated in the horizontal plane. As the three-dimensional location of the trans-esophageal ultrasonic probe also is referenced to the same external reference transducers, the methodology of the present invention provides a more robust application. Image mapping is enhanced because the image data acquired at the second location can be combined with other acquired image data and used to perform a 3D reconstruction on any structure being imaged.

In more particular embodiments, the methodology of the present invention includes selecting one location for imaging a given body part from one vantage point and selecting another location for imaging the given body part from another vantage point. Thereafter, said step of navigating another probe and generating three dimensional coordinates (Step 104), generating image date for a given body part or a portion when it is determined that the probe or instrument is at the second or another location (Steps 106, 108), and registering the image date for the given body part with the three dimensional coordinates of the second or another body location (Step 110) are performed when the ultrasonic probe is located at each of the selected locations.

In further embodiments, the body part is the heart and according to the methodology of the present invention the first body location from which the heart is to be imaged is within the circulatory system of the body and the second body location from which the heart is to be imaged is within the circulatory system of the body, however, it is remote from the first location.

In yet further embodiments, the body part is the heart and according to the methodology of the present invention the first body location from which the heart is to be imaged is within the circulatory system of the body and the second body location from which the heart is to be imaged is outside of the circulatory system of the body and remote from the first location.

In yet further embodiments, the body part is the heart and according to the methodology of the present invention the first body location from which the heart is to be imaged is within the circulatory system of the body being imaged and the second body location from which the heart is to be imaged is within the esophagus.

In yet further embodiments of the methodology of the present invention, the ultrasonic probe within the circulatory system is an intracardiac ultrasound catheter and the ultrasonic probe within the esophagus is a trans-esophageal ultrasonic probe.

If it is determined that additional imaging is not to be performed at a different location or second location (Step 112, No) a process is performed to reconstruct a 3-D image of the given body part using the image data acquired at each of the first and second locations and the three-dimensional coordinate data generated for each of the first and second locations, Step 114. In the case where image data is acquired from n locations, the 3-D image of the given body part is reconstructed using the image data acquired at each of the n locations and the three-dimensional coordinate data generated for each of n locations.

In yet further embodiments, image data is acquired using different imaging modalities. In such a case, the 3-D image is reconstructed using the image data obtained from each imaging modality. If needed, the three-dimensional coordinates of a fiducial visible in an imaging modality are determined using the methods of the present invention and the fiducial is used to register the imaging data of the another imaging modality to the determined three dimensional coordinates.

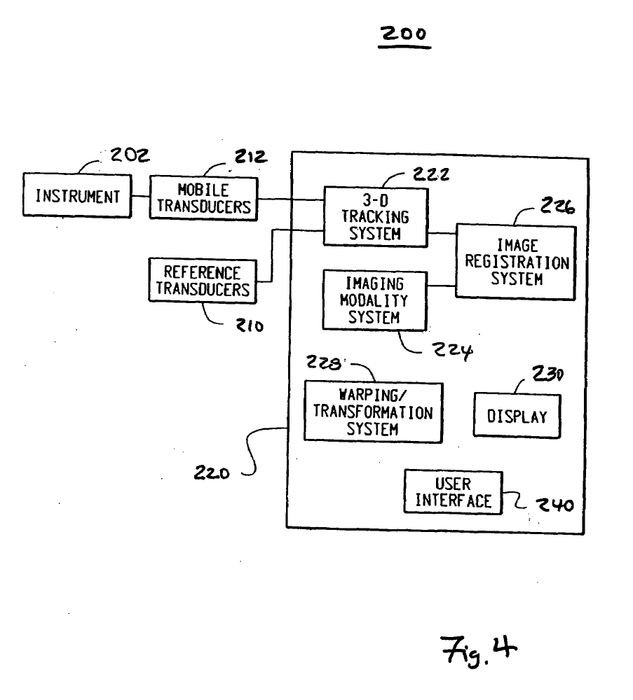

Referring now to FIG. 4 there is shown an illustrative exemplary embodiment of a 3-D tracking and imaging system 200 according to the present invention. Such a 3-D tracking and imaging system 200 is generally comprised of a computer system 220, one or more mobile transducers 212, one or more reference transducers 210, and an instrument 202 or probe.

The computer system 220 is generally comprised of a 3-D tracking system 222, an imaging modality system 224, an image registration system 226, a user interface 240 and a display 230. It should be appreciated that 3-D tracking system 222 can take the form of a sound-based system or an electromagnetic-based system. Both time of flight and phase relationships may be used to determine distance.

The instrument 202 can take the form of a catheter, a probe, a sensor, a needle, a scalpel, a forceps or other device used in a surgical or diagnostic procedure. Mobile transducers 212 and reference transducers 210 can take the form of an ultrasonic transducer or an electronic transducer. For purpose of illustrating embodiments of the present invention, however, the reference and mobile and transducers 210,212 will take the form of ultrasonic transducers (i.e., piezoelectric crystals) such as those described herein.

As indicated herein one or more mobile transducers 212 are fitted to the instrument 202. The one or more reference transducers 210 provide a reference position relative to mobile transducers 202. In this respect, the reference transducers 210 are located so as to provide an internal reference frame inside a patient's body or on the surface of a patient's body to provide an external reference frame. As indicated herein, the reference transducers 210 can be transmitters, transceivers or receivers that can generate ultrasound or electromagnetic radiation, that can be detected by the mobile transducers 212.

For the purpose of illustration the 3-D tracking system 222 takes the form of the ultrasonic 3-D tracking system described herein and the references identified herein. Such a 3-D tracking system 222 transforms the multiple distance measurements between all of the transducers 210,212 into XYZ coordinates relative to a referenced axis. It should be appreciated that the reference frame provided by the reference transducers 210 is self-determining, that is, if the reference frame becomes distorted, this distortion needs to be detected by reference transducers. Detection is typically done by using transceivers that can determine the distance between any combination of two transducers, and hence their relative spacial coordinates in 3-D space. In this regard, the position of the transducers is obtained in 3-D from the images acquired of the bodily structure (e.g., tissue/organ) that show “dots” where the transducers are located, and also from the transducers themselves when they are in the bodily structure. If there is some discrepancy in the distances between all combinations of transducers, then the bodily structure must have deformed (i.e., “warped”) after the images were acquired. A mathematical coordinate transformation can be used to specify exactly how to correct the image set and account for the warping, for example, using the warping/transformation system 228. The distance between any combination of two transducers is determined by having each transducer send a signal to all other transducers. In this way, all the distances between the transducers is known. From these distances, XYZ coordinates can be calculated, in reference to some transducer as the origin.

The imaging modality system 224 acquires 2-D, 3-D or 4-D image data sets from an imaging source, such as fluoroscopy, an MRI (magnetic resonance imaging), CT (computerized tomography) or 2-D or 3-D ultrasound device, to provide a “template” through or against which the shape, position and movement of instrument 202 being tracked can be displayed. The template typically takes the form of an image of the environment surrounding the instrument (e.g., a bodily structure). It should be noted that if multiple (3-D) volumes are acquired at different time intervals, a 4-D image is obtained (i.e., 3-D image changing over time).

The image registration system 226 registers the position of the instrument 202 within the spatial coordinates of the image data set provided by imaging modality system 224. The position of instrument 202 is provided by the 3-D tracking system 222. The image registration system 226 will provide a display of instrument 202 at its proper 3-D location inside the bodily structure and orientation relative to the bodily structure itself. It should be appreciated that the registration system 226 can be user assisted, or completely automated if image processing algorithms are implemented to automatically detect the spatial locations of the transducers (typically the reference transducers) in the image data set.

The user interface 240 enables a user to interact with the computer system 220, including programming the computer system 220 to perform a desired function. For example, a particular view for display can be selected. The instrument(s) 202 (e.g., probes or catheters) can be activated using the user interface 230. The display 230 is any of a number of display devices known in the art, so as to display to the user registered images provided by image registration system 226.

As discussed above, the 3-D tracking and imaging system 200 can display existing or user acquired image data sets as a template through which, or against which the position, shape or motion of an instrument 202 can be referenced inside the body or organ. This can be accomplishes using any of a number of algorithms as is known to those skilled in the art or hereinafter developed.

Although a preferred embodiment of the invention has been described using specific terms, such description is for illustrative purposes only, and it is to be understood that changes and variations may be made without departing from the spirit or scope of the following claims.

### INCORPORATION BY REFERENCE

All patents, published patent applications and other references disclosed herein are hereby expressly incorporated by reference in their entireties by reference.

### EQUIVALENTS

Those skilled in the art will recognize, or be able to ascertain using no more than routine experimentation, many equivalents of the specific embodiments of the invention described herein. Such equivalents are intended to be encompassed by the following claims.

What is claimed is:

1. A method for imaging tissue using ultrasonic imaging techniques and a 3-D tracking system; said method comprising the steps of:
mounting at least one transducer to an ultrasonic probe;
mounting one or more reference ultrasonic transducers to locations on the body to be imaged, where the one or more reference transducers each have a position fixed relative to the body during the imaging process;
generating three dimensional coordinates of the at least one transducer mounted to the ultrasonic probe relative to a reference frame established by the one or more reference transducers;
generating image data using the ultrasonic probe; and
registering the three-dimensional coordinates with the image data from the ultrasonic probe to form a 3-D image scene.

2. The method of claim 1, further comprising the steps of:
performing said steps of generating three dimensional coordinates, generating image date for given body part and registering when the ultrasonic probe is located at a first location;
performing said steps of generating three dimensional coordinates, generating image date for the given part and registering when another ultrasonic probe is located at a second body location; and
wherein the generating of three dimensional coordinates at the first and second locations being generated using the same one or more reference transducers and the second location being remote from the first location.

3. The method of claim 2, wherein said steps of generating three dimensional coordinates, generating image date for given body part and registering are performed N times, at N different locations of the ultrasonic probe.

4. The method of claim 1, further comprising the step of:
selecting one location for imaging a given body part from one vantage point and selecting another location for imaging the given body part from another vantage point; and
performing said steps of generating three dimensional coordinates, generating image date for given body part and registering when the ultrasonic probe is located at each of the selected locations.

5. The method of claim 2, wherein said first and second locations are selected so that the given body part is imaged from a first vantage point as the first location and is imaged from a second vantage point as the second location.

6. The method of claim 2, wherein:
the body part is the heart;
the first body location from which the heart is to be imaged is within the circulatory system of the body being imaged; and
the second body location from which the heart is to be imaged is within the circulatory system of the body and remote from the first location.

7. The method of claim 2, wherein:
the body part is the heart;
the first body location from which the heart is to be imaged is within the circulatory system of the body being imaged; and
the second body location from which the heart is to be imaged is outside of the circulatory system of the body and remote from the first location.

8. The method of claim 2, wherein:
the body part is the heart;
the first body location from which the heart is to be imaged is within the circulatory system of the body being imaged; and
the second body location from which the heart is to be imaged is within the esophagus.

9. The method of claim 8, wherein:
the ultrasonic probe within the circulatory system is an intracardiac ultrasound catheter; and
the ultrasonic probe within the esophagus is a trans-esophageal ultrasonic probe.

10. The method of claim 2, further comprising the steps of:
performing a 3-D reconstruction of the given body part using the image data acquired at each of the first and second locations and the three-dimensional coordinate data generated for each of the first and second locations.

11. The method of claim 8, further comprising the steps of:
performing a 3-D reconstruction of the heart using the image data acquired at each of the first and second locations and the three-dimensional coordinate data generated when the ultrasonic probe is within the circulatory system and when the another ultrasonic is within the esophagus.
